(19) United States
(12) Patent Application Publication
(10) Pub. No.: US 2022/0230735 A1
(43) Pub. Date: Jul. 21, 2022

(54) METHODS AND SYSTEMS FOR INTEGRATING CLINICAL PATHWAY MANAGEMENT SYSTEMS WITH ELECTRONIC MEDICAL RECORD DATABASES

(71) Applicant: KONINKLIJKE PHILIPS N.V., Eindhoven (NL)

(72) Inventors: Ricardo da Silva Santos, São Paulo (BR); Natasha Markuzon, Cambridge, MA (US)

(21) Appl. No.: 17/150,240

(22) Filed: Jan. 15, 2021

(51) Int. Cl. G16H 40/20; G16H 10/60; G06F 16/242

(52) U.S. Cl. CPC G16H 40/20; G06F 16/2433; G16H 10/60

(57) ABSTRACT

A method for integrating clinical pathway management systems with electronic medical record databases, comprising: (i) providing a CPMS database comprising information about a current clinical pathway for patients; (ii) providing an electronic medical records (EMR) database comprising medical records for the patients; (iii) generating an EMR equivalence map comprising a map of each of objects in the EMR database to standard data format objects; (iv) generating a CPMS equivalence map for the CPMS database, comprising a map of objects in the CPMS database to the standard data format objects; (v) mapping objects from the EMR equivalence map to each of one or more steps in one of a plurality of clinical pathway workflows; (vi) generating a SQL command related to an event in a clinical pathway workflow for one or more of the plurality of patients; and (vii) executing the generated SQL command to retrieve an electronic medical record from the EMR database.

### FIELD OF THE DISCLOSURE

The present disclosure is directed generally to methods and systems for integrating clinical pathway management systems with electronic medical record databases, thereby enabling real-time monitoring of clinical pathways execution.

### BACKGROUND

A clinical pathway (CP), also known as a care pathway or clinical protocol, is a tool utilized in the healthcare system to standardize care, in part by minimizing variations in care and treatment procedures for a specific disease. A CP can also be described as a multidisciplinary management tool based on evidence-based practice for a specific group of patients with a predictable clinical course, in which the different tasks or interventions by the professionals involved in the patient care are defined, optimized and sequenced by intervals such as hour, day or visit.

Numerous studies have demonstrated that implementation of clinical pathways reduce variability and improve patient outcomes in many different healthcare settings. Despite studies showing the benefits of clinical pathway implementation, utilization of and compliance with clinical pathway systems is generally lower than desired or expected by healthcare quality analysts.

Clinical pathways management systems (CPMS) are software tools that implement clinical pathways and therefore assistant healthcare professionals adhere to these clinical pathways by identifying deviations in the workflow and emitting alerts to the healthcare professionals. Indeed, ensuring clinical pathway compliance by managing its execution is essential, and CPMS are a vital tool to track clinical pathway execution. Existing CPMS neglect the integration of CPMS with electronic medical record databases (EMR), and the few that address the topic do not propose an easy and flexible integration method.

### SUMMARY OF THE DISCLOSURE

There is a continued need for methods and systems that suitably integrate clinical pathways management systems (CPMS) with electronic medical record databases (EMR). Various embodiments and implementations herein are directed to a method and system configured to integrate CPMS with EMR. The system comprises: (i) an EMR database comprising one or more medical records for each of a plurality of patients currently undergoing care in a clinical pathway; and (ii) a CPMS database comprising information about a current clinical pathway for each of the plurality of patients currently undergoing care. The system generates an EMR equivalence map for the EMR database comprising a map of each of a plurality of objects in the EMR database to each of a plurality of fast healthcare interoperability resources (FHIR). The system also generates a CPMS equivalence map comprising a map of each of a plurality of objects in the CPMS database to the plurality of FHIR. The system maps each of one or more objects from the EMR equivalence map to each of one or more steps in one of a plurality of clinical pathway workflows and generates, using the map, a SQL command related to an event in a clinical pathway workflow for one or more of the plurality of patients. The SQL command is then executed to retrieve an electronic medical record from the EMR database. According to an embodiment, the system analyzes the retrieved electronic medical record for a deviation from a predefined clinical pathway workflow for one or more patients. The system also analyses the retrieved electronic medical record for a bottleneck in a predefined clinical pathway workflow currently being executed for the patient. An alert is generated if there is a deviation and/or bottleneck identified, and the alert is provided via a user interface.

Generally, in one aspect, a method for integrating clinical pathway management systems with electronic medical record databases is provided. The method includes: (i) providing a clinical pathways management system (CPMS) comprising a CPMS database, the CPMS database comprising information about a current clinical pathway for each of the plurality of patients currently undergoing care; (ii) providing an electronic medical records (EMR) database comprising one or more medical records for each of a plurality of patients currently undergoing care in a clinical pathway; (iii) generating in memory an EMR equivalence map for the EMR database, the EMR equivalence map comprising a map of each of a plurality of objects in the EMR database to each of a plurality of standard data format objects; (iv) generating in memory a CPMS equivalence map for the CPMS database, the CPMS equivalence map comprising a map of each of a plurality of objects in the CPMS database to the plurality of standard data format objects; (v) mapping each of one or more objects from the EMR equivalence map to each of one or more steps in one of a plurality of clinical pathway workflows; (vi) generating, using the map of each of one or more objects from the EMR equivalence map to each of one or more steps in one of a plurality of clinical pathway workflows, a SQL command related to an event in a clinical pathway workflow for one or more of the plurality of patients; and (vii) executing the generated SQL command to retrieve an electronic medical record from the EMR database.

According to an embodiment, the method further includes the steps of analyzing, for at least one of the one or more of the plurality of patients, the retrieved electronic medical record for a deviation from a predefined clinical pathway workflow; generating an alert if there is a deviation identified; and providing, via a user interface, a visualization of the identified deviation. According to an embodiment, the visualization of the identified deviation is provided via a mobile application. According to an embodiment, the identified deviation is provided via a mobile application utilizing a green, yellow, and red code.

According to an embodiment, the method further includes the steps of analyzing, for at least one of the one or more of the plurality of patients, the retrieved electronic medical record for a bottleneck in a predefined clinical pathway workflow currently being executed for the at least one patient; generating an alert if there is a bottleneck identified; and providing, via a user interface, a visualization of the identified bottleneck. According to embodiment, the visualization of the identified bottleneck is provided via a mobile application. According to an embodiment, the bottleneck is a time bottleneck and/or a resource bottleneck.

According to an embodiment, the standard data format objects are fast healthcare interoperability resources (FHIR).

According to an embodiment, the map of each of one or more objects from the EMR equivalence map to each of one or more steps in one of a plurality of clinical pathway workflows comprises Business Process Model and Notation (BPMN).

According to another aspect is a system for integrating clinical pathway management systems with electronic medical record databases. The system comprises: a clinical pathways management system (CPMS) comprising a CPMS database, the CPMS database comprising information about a current clinical pathway for each of the plurality of patients currently undergoing care; an electronic medical records (EMR) database (270) comprising one or more medical records for each of a plurality of patients currently undergoing care in a clinical pathway; an EMR equivalence map (263) for the EMR database, the EMR equivalence map comprising a map of each of a plurality of objects in the EMR database to each of a plurality of standard data format objects; a CPMS equivalence map (263) for the CPMS database, the CPMS equivalence map comprising a map of each of a plurality of objects in the CPMS database to the plurality of standard data format objects; and a processor (220) configured to: (i) map each of one or more objects from the EMR equivalence map to each of one or more steps in one of a plurality of clinical pathway workflows; (ii) generate, using the map of each of one or more objects from the EMR equivalence map to each of one or more steps in one of a plurality of clinical pathway workflows, a SQL command related to an event in a clinical pathway workflow for one or more of the plurality of patients; and (iii) execute the generated SQL command to retrieve an electronic medical record from the EMR database.

According to an embodiment, the processor is further configured to: analyze, for at least one of the one or more of the plurality of patients, the retrieved electronic medical record for a deviation from a predefined clinical pathway workflow; generate an alert if there is a deviation identified; and direct the system to provide, via a user interface, a visualization of the identified deviation. According to an embodiment, the system further comprises a mobile application with the user interface configured to provide the visualization of the identified deviation.

According to an embodiment, the processor is further configured to: analyze, for at least one of the one or more of the plurality of patients, the retrieved electronic medical record for a bottleneck in a predefined clinical pathway workflow currently being executed for the at least one patient; generate an alert if there is a bottleneck identified; and direct the system to provide, via a user interface, a visualization of the identified bottleneck. According to an embodiment, the system further comprises a mobile application with the user interface configured to provide the visualization of the identified bottleneck.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for integrating clinical pathway management systems with electronic medical record databases, in accordance with an embodiment.

FIG. 2 is a schematic representation of an integration system, in accordance with an embodiment.

FIG. 3 is a schematic representation of an integration system, in accordance with an embodiment.

FIG. 4 is a schematic representation of an entity relationship diagram for a CPMS database, in accordance with an embodiment.

FIG. 5 is a schematic representation of a portion of a clinical pathway BPMN diagram, in accordance with an embodiment.

FIG. 6 is a schematic representation of a configuration interface, in accordance with an embodiment.

FIG. 7 is a flowchart of a method for generating a SQL command, in accordance with an embodiment.

FIG. 8 is a schematic representation of relationships established for FHIR resources, in accordance with an embodiment.

FIG. 9 is a schematic representation of an integration system, in accordance with an embodiment.

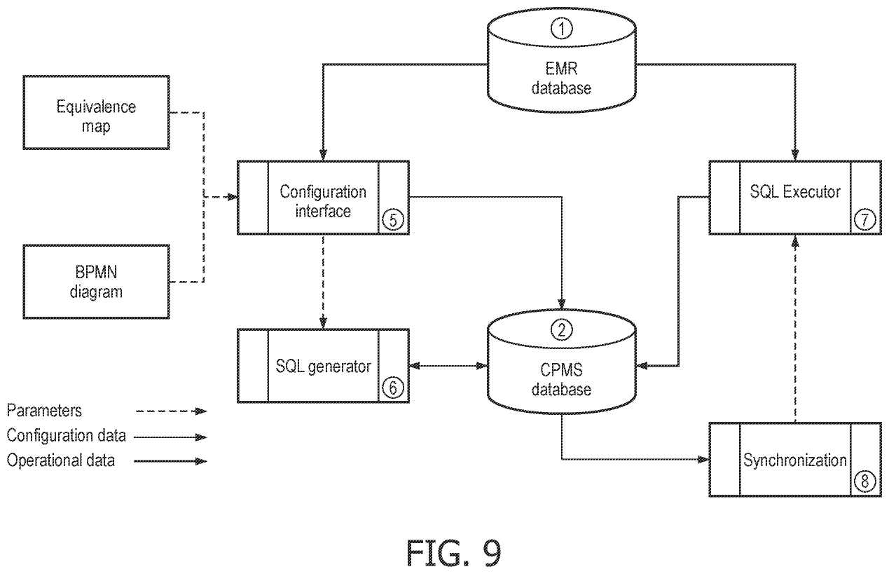

FIG. 10 is a flowchart of a method for integrating clinical pathway management systems with electronic medical record databases, in accordance with an embodiment.

FIG. 11 is a schematic representation of a CPMS Server, in accordance with an embodiment.

FIG. 12 is a schematic representation of a Protocol Analyzer, in accordance with an embodiment.

FIG. 13 is a schematic representation of a CPMS Client Application, in accordance with an embodiment.

FIG. 14 is a schematic representation of a CPMS Client Application visualization, in accordance with an embodiment.

FIG. 15 is a schematic representation of a Patient Status Visualizer, in accordance with an embodiment.

FIG. 16 is a schematic representation of a Bottlenecks Visualizer, in accordance with an embodiment.

### DETAILED DESCRIPTION OF EMBODIMENTS

The present disclosure describes various embodiments of a system and method configured to integrate clinical pathway management systems with electronic medical record databases, and optionally alerting a user of a clinical pathways management system to a clinical pathway deviation and/or bottleneck. More generally, Applicant has recognized and appreciated that it would be beneficial to provide a method and system to integrate clinical pathway management systems with electronic medical record databases and monitor patient care. The system generates an EMR equivalence map for an EMR database comprising a map of each of a plurality of objects in the EMR database to each of a plurality of fast healthcare interoperability resources (FHIR). The system also generates a CPMS equivalence map comprising a map of each of a plurality of objects in a CPMS database to the plurality of FHIR. The system maps each of one or more objects from the EMR equivalence map to each of one or more steps in one of a plurality of clinical pathway workflows and generates, using the map, a SQL command related to an event in a clinical pathway workflow for one or more of the plurality of patients. The SQL command is then executed to retrieve an electronic medical record from the EMR database. According to an embodiment, the system analyzes the retrieved electronic medical record for a deviation from a predefined clinical pathway workflow for one or more patients. The system also analyses the retrieved electronic medical record for a bottleneck in a predefined clinical pathway workflow currently being executed for the patient. An alert is generated if there is a deviation and/or bottleneck identified, and the alert is provided via a user interface

In certain embodiments, healthcare professionals use information about clinical pathway deviations and/or bottlenecks provided by the clinical pathways management system for treatment of a patient. For example, identification of a clinical pathway deviation by the system via an alert or other notification method can not only prevent errors or unwanted/unnecessary treatment, but can cause the healthcare professional to modify a patient's care. The healthcare professional may utilize the information about the deviation to act in the task that led to the deviation, such that there is no longer a deviation. Alternatively, the healthcare professional may treat the patient in another way to rectify or obviate the deviation or effects of the deviation. Many other responding effects resulting from the identification of a deviation are possible. Similarly, identification of a bottleneck in treatment can lead to amelioration of that bottleneck, therefore leading to improved care for one or possibly multiple patients.

Accordingly, the methods and systems described or otherwise envisioned herein provide a method to integrate CPMS with EMR to enable real-time monitoring of clinical pathways execution. In certain embodiments, healthcare institutions use information about clinical pathway deviations and/or bottlenecks provided by the clinical pathways management system to analyze or evaluate care at that institution. For example, the institution may gather statistics for deviations and/or bottlenecks by an individual, a department, a treatment, a patient, an organization, and/or at many other levels or groupings. This may help the institution identify problem areas such as individuals, departments, treatments, and more. The institution may then be able to address the problem area utilizing the analysis provided by the clinical pathway management system.

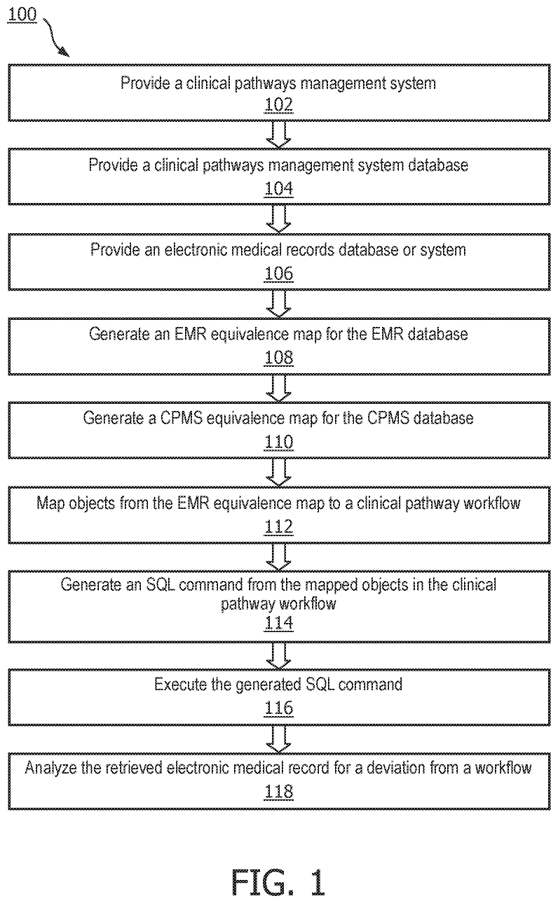

Referring to FIG. 1, in one embodiment, is a flowchart of a method 100 for integrating clinical pathway management systems with electronic medical record databases. The methods described in connection with the figures are provided as examples only, and shall be understood not to limit the scope of the disclosure. The integration system, the clinical pathways management system, and the electronic medical records system can be any of the systems described or otherwise envisioned herein. The integration system, the clinical pathways management system, and the electronic medical records system can be a single system or multiple different systems.

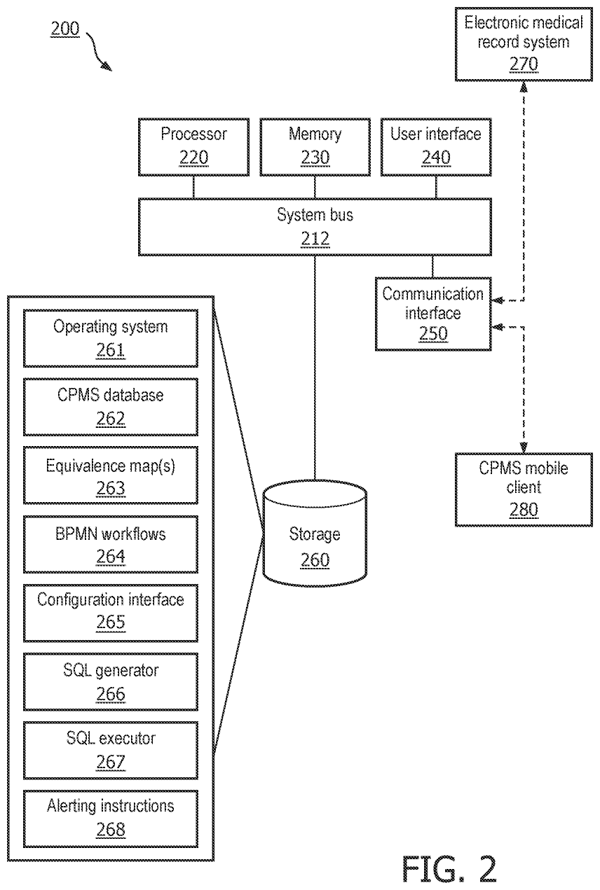

At step 102 of the method, an integration system 200 is provided. Referring to an embodiment of a integration system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, integration system 200 can be any of the systems described or otherwise envisioned herein. Other elements and components of the integration system 200 are disclosed and/or envisioned elsewhere herein.

According to an embodiment, a clinical pathways management system comprises digital patient care pathways that optimize the delivery of treatment to patients. The system is an important tool that supports clinical pathway execution by, among other things, providing pathway information, checking pathway compliance, facilitating communication, and more. The system can, for example, provide an overview of the compliance status of all clinical pathways running in a care facility, a department, and/or other care breakdowns. The clinical pathways management system can provide many other functions to facilitate patient care.

At step 104 of the method, a clinical pathways management system database (CPMS database) is provided. According to an embodiment, the clinical pathways management system 200 comprises a CPMS database 262 comprising information about a current clinical pathway for each of the plurality of patients currently undergoing care. Alternatively, the CPMS database is hosted or stored remotely and is in communication the integration system and/or system 200. According to an embodiment, the CPMS database contains data related to details of clinical pathways execution, such as an event log, among other possible data.

At step 106 of the method, an electronic medical record database or system (EMR database) is provided. The electronic medical record database or system comprises, among other possible data, one or more medical records for each of a plurality of patients currently undergoing care in a clinical pathway. According to an embodiment, the integration system comprises an electronic medical record database or system 270 which is optionally in direct and/or indirect communication with the clinical pathways management system 200.

According to an embodiment, the integration system integrates the CPMS and the EMR database, while focusing on flexibility, usability, and performance. Flexibility is the ability to integrate CPMS with different EMRs, by using of a universal data exchange protocol, such as HL7, as discussed below. The usability of the method is provided by the ease of configuration, allowing even a user without a technical background be able to define the integration with a very short training time. Additionally, the mechanism of data retrieving defined in the method allows data synchronization between the CPMS and the EMR without intermediate layers in order to ensure enough performance for real-time monitoring of clinical pathways execution.

Accordingly, at step 108 of the method the integration system generates an EMR equivalence map for the EMR database. According to an embodiment, the EMR equivalence map comprises a map of each of a plurality of objects in the EMR database to each of a plurality of elements in a standard data format. According to an embodiment, that standard data format can be fast healthcare interoperability resources (FHIR). FIHR, created by the Health Level Seven International (HL7) health-care standards organization, is a standard that describes data formats and elements (also called resources), and provides an API to exchange electronic health records. FHIR enables interoperation between health care systems by facilitating communication between systems and enabling the use of many different devices, and allows third-party application developers to create applications that can then be integrated into existing health record systems. According to an embodiment, the EMR database is compatible with the standard data format, such as the FHIR format. The more compatible the EMR database is to the standard, such as to the HL7 protocol, the less effort needed to configure the integration. For example, an EMR database may have one table for each FIHR resource. In this situation, no additional effort will be required for integration mapping. On the other hand, if an EMR database stores data related to a specific FIHR resource in several tables, the development of a database view for consolidating these tables can be necessary before the integration mapping.

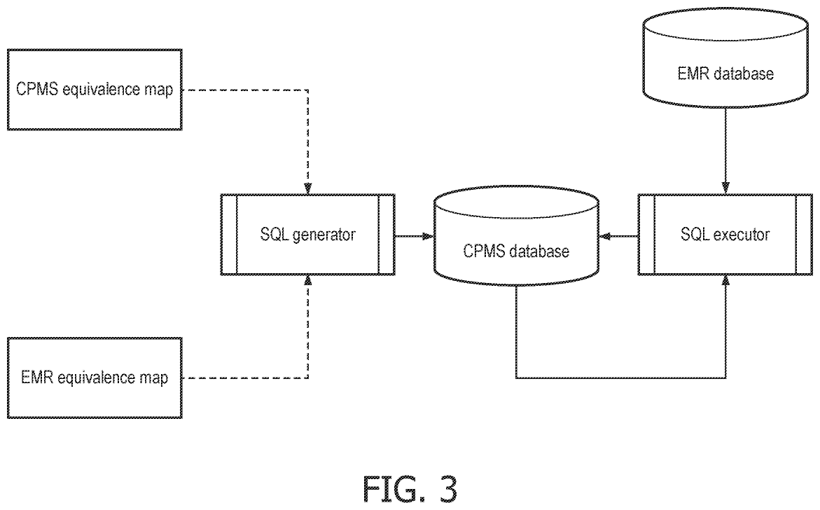

According to an embodiment, the EMR equivalence map comprises all information required by a SQL generator (described elsewhere herein) to create SQL commands, which will be executed by a SQL executor, thus completing the basic integration process as shown in FIG. 3. According to an embodiment, the equivalence map comprises or is implemented by two distinct maps. The integration system identifies which FIHR resources match to the objects of the target database, the CPSM, and then, using this FIHR resources list, the system matches in the source database, the EMR, which objects corresponding to the each FHIR resources of the list. TABLE 1 is an example of an equivalence map for the EMR database, and TABLE 2 is an example of an equivalence map for the CPMS database. The equivalence map for the EMR database may be the one that is utilized for generating SQL commands, so it may contain some additional details such as the table primary key (PK). Despite the usability of the configuration interface, the equivalence maps can be defined by a technical professional who should has a deep knowledge regarding the EMR database schema.

TABLE 1
Sample Equivalence Map for EMR database

| | | EMR | | |
|---|---|---|---|---|
| HL7 FHIR | | Database | | |
| Resources | Attribute | Table | PK | Column |
| EpisodeOfCare | | | | |
| | Identifier | | | |
| | Period (begin) | | | |
| | Period (end) | | | |
| Encounter | | | | |
| | .Hospitalization | | | |
| | (Origin) | | | |
| | .Hospitalization | | | |
| | (Destination) | | | |
| | .Hospitalization | | | |
| | (dischargeDisposition) | | | |
| | Status | | | |
| Condition | | | | |
| | ClinicalStatus | | | |
| Observation | | | | |
| | Identifier | | | |
| | Category | | | |
| | Effective. | | | |
| | effectiveDateTime | | | |
| | Value.valueQuantity | | | |
| | referencerange | | | |
| | interpretation | | | |
| Task | | | | |
| | Identifier | | | |
| | ExecutionPeriod | | | |
| Procedure | | | | |
| | Identifier | | | |
| | PerformedDateTime | | | |
| | Note | | | |
| ProcedureRequest | | | | |
| | Identifier | | | |
| | authoredOn | | | |
| | Occurrence | | | |
| | Note | | | |
| MedicationRequest | | | | |
| | Identifier | | | |
| | authoredOn | | | |
| | Occurrence | | | |
| | Dosage | | | |
| | Note | | | |
| MedicationAdministration | | | | |
| | Identifier | | | |
| | effectiveDateTime | | | |
| | Note | | | |
| PractitionerRole | | | | |
| | Code | | | |

At step 110 of the method, the integration system generates a CPMS equivalence map for the CPMS database. According to an embodiment, the CPMS equivalence map comprises a map of each of a plurality of objects in the CPMS database to the plurality of FIHR resources.

According to an embodiment, the CPMS database is the destination of the integration between the CPMS and the electronic medical record database. The CPMS database can store an event log of clinical pathways execution, as well as one or more configuration parameters of the integration. According to an embodiment, the design of a CPMS database can be guided by HL7 FHIR concepts, although other standards are possible. Notably, this does not mean that the database objects are exactly the same as the FHIR resources. According to an embodiment, each database column in the CPMS database has a corresponding attribute in a FHIR resource. In this way, the integration system can properly express the event log for the CPMS and ensure an easy integration with different EMR databases as described or otherwise envisioned herein.

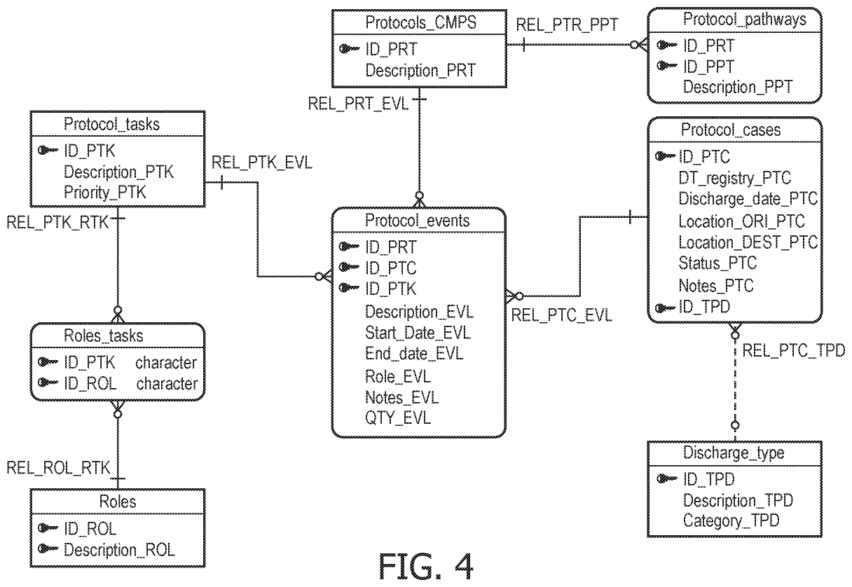

Referring to FIG. 4, in one embodiment, is an entity relationship diagram for the CPMS database, with reference to TABLE 2, which is an equivalence map for the CPMS. According to just one embodiment, two tables of CPMS database will receive data continuously from the EMR, namely PROTOCOL_CASES and PROTOCOL_EVENTS. For example, PROTOCOL_CASES is equivalent to the FHIR resources EpisodeOfCare and Encounter. These resources contains basic data regarding each case included in the treatment protocol. The PROTOCOL_EVENTS table stores data regarding execution of the clinical pathway tasks. According to an embodiment, each record of TABLE 2 corresponds to one of the following FHIR resources: ProcedureRequest; MedicationRequest; Procedure; MedicationDispense; MedicationAdministration. The other tables of CPMS database store auxiliary data of clinical pathway management process, such as the protocols managed by the hospital, the tasks of each protocol, the roles allowed for each task, and many other possible data.

TABLE 2
Sample Equivalence Map for CPMS

| CPMS Database Columns | FHIR Resource Attributes |
|---|---|
| PROTOCOL_CASES | EpisodeOfCare -> identifier |
| ID_PTC | EpisodeOfCare -> Period (begin) |
| DT_REGISTRY_PTC | EpisodeOfCare -> Period (end) |
| DICHARGE_DATE_PTC | Encounter -> Hospitalization (Origin) |
| LOCATION_ORI_PTC | Encounter -> Hospitalization (Destination) |
| STATUS_PTC | Encounter -> Status |
| NOTES_PTC | Condition -> clinical Status |
| ID_TPD | Encounter -> Hospitalization (dischargeDisposition) |
| PROTOCOL_EVENTS | |
| ID_PTK | X -> Identifier |
| DESCRIPTION_EVL | Task -> description |
| START_DATE_EVL | ProcedureRequest -> Occurrence |
| | MedicationRequest -> Occurrence |
| | Procedure -> PerformedDateTime |
| | MedicationDispense -> whenPrepared |
| | MedicationAdministration -> effectiveDateTime |
| END_DATE_EVL | ProcedureRequest -> authoredOn |
| | MedicationRequest -> authoredOn |
| | Procedure -> PerformedDateTime |
| | MedicationDispense -> whenHandedOver |
| | MedicationAdministration -> effectiveDateTime |
| ROLE_EVL | PractitionerRole -> Code |
| NOTES_EVL | X -> note |
| QTY_EVL | MedicationRequest -> Dosage |
| | MedicationDispense -> quantity |

X = ProcedureRequest; MedicationRequest; Procedure; MedicationDispense; MedicationAdministration

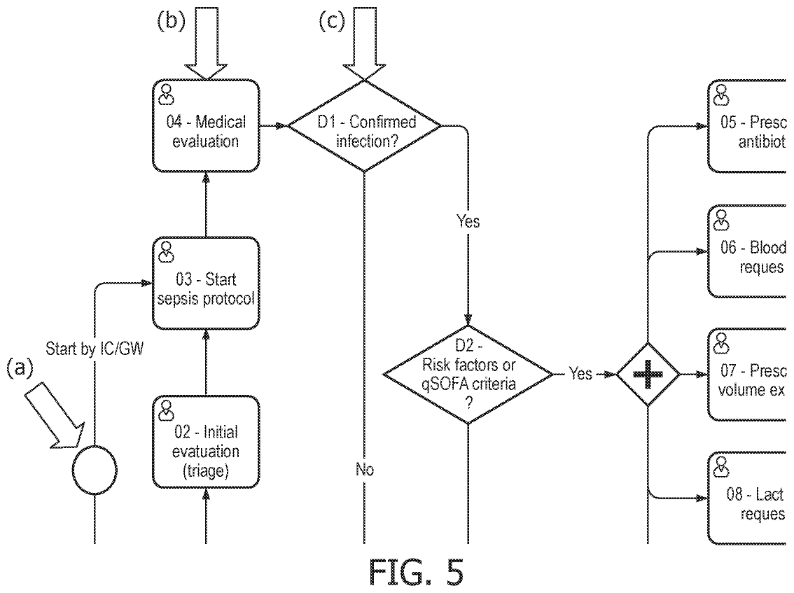

At step 112 of the method, according to an embodiment, the integration system maps each of one or more objects from the EMR equivalence map to each of one or more steps in one of a plurality of clinical pathway workflows. Therefore, according to an embodiment, the integration system comprises one or more digital workflows to which objects can be mapped. For example, the integration system may comprise a business process model and notation (BPMN) representation specifying one or more workflows or processes. The data transferred from the EMR to the CPMS can be related to the execution of tasks of a BPMN diagram. In this way, the integration system may allow the configuration of each element of the BPMN diagram. In the context of clinical pathway management systems, three elements of a BPMN diagram can be monitored: (a) start event, (b) tasks and (c) gateways. A portion of a clinical pathway BPMN diagram is shown in FIG. 5 with reference to these three elements.

According to an embodiment, the start event indicates the trigger for inclusion of patient into the protocol. This event, in practice, can be the execution of a particular task (including any medical procedures) or a patient's clinical condition (defined by the result of any diagnosis exams). Therefore, the following FIHR resources can be used for configuration of the start event: ProcedureRequest; MedicationRequest; MedicationDispense; MedicationAdministration; Observation and Task, among others. According to an embodiment, a task in the BPMN diagram indicates any activity performed on the patient's treatment, whether this is clinical activity (e.g., medication administration) or not (e.g., patient label checking). Thus, all FIHR resources used in the configuration of the start event, except Observation, can also be used for configuration of a BPMN task. According to scope of the CPMS, a gateway BPMN is used to indicate the patient's clinical status or the completion status of a task. Thus, the FIHR resource that can be used for configuration of this element may be Observation.

According to an embodiment, the integration system comprises a configuration interface that enables the user, without any programming effort, to configure the data integration between the ERM and the CPMS, thus integrating each clinical pathway BPMN element.

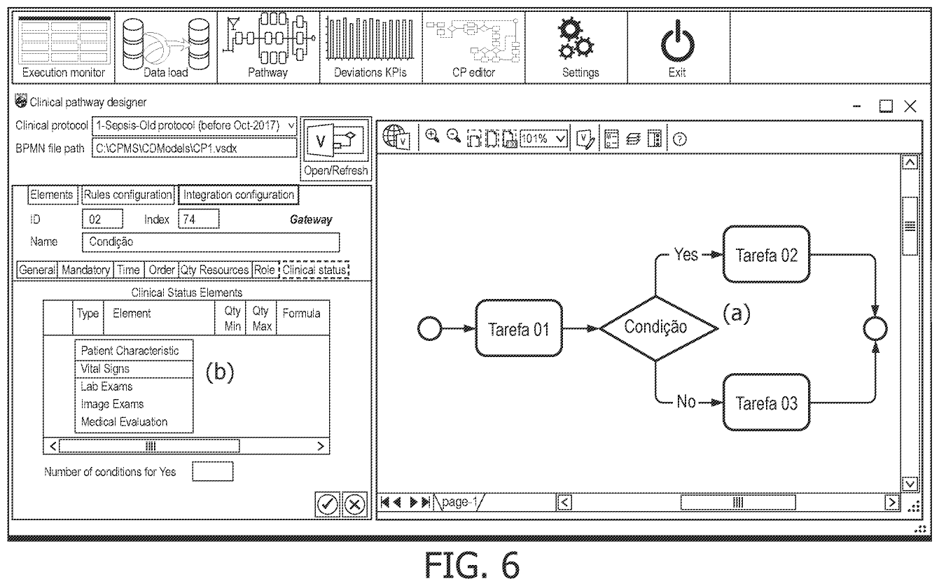

Referring to FIG. 6 is an embodiment of a configuration interface, which is an interface that allow the configuration of the integration just by selecting the objects from EMR database and linking them to the elements of BPMN workflow. According to an embodiment, the operation of the configuration interface consists of to select a BPMN element on the right side of the screen and to indicate, on the left side, which EMR database object should provide the data for that BPMN element. This operation is possible due to the equivalence map. Since the equivalence map defines all valid EMR tables and columns for a particular FHIR resource, the configuration interface may create a drop-down list with the database objects that the user can link to that BPMN element, according to an embodiment.

As just one example, consider the configuration of a particular gateway of the BPMN diagram. According to the proposed method, a gateway corresponds to the FHIR resource Observation. Thus, a drop-down list will display all EMR database tables that were assigned to this FHIR resource. Accordingly, the user need just indicate one or more tables that will be the data source from that BPMN element. Table columns does not need to be indicated at this time of configuration, since they have already been mapped in the equivalence map.

According to an embodiment, in addition to selecting tables, the user can include filters in the configuration. Consider, for example, that the user is configuring a gateway to validate the following condition. If Patient temperature>30, then output=Yes. Otherwise, output=No. In this case, in addition to the user selecting the “Vital Signs” table, it must include a filter for the “Temperature” value. This means that at the time of data synchronization, only the records with value “Temperature” should be retrieved.

At step 114 of the method, according to an embodiment, the integration system generates, using the map of each of one or more objects from the EMR equivalence map to each of one or more steps in one of a plurality of clinical pathway workflows, a SQL command related to an event in a clinical pathway workflow for one or more of the plurality of patients.

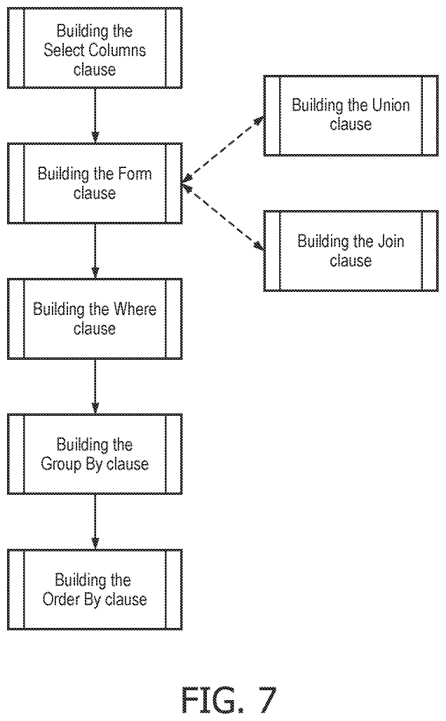

Referring to FIG. 7 is a flowchart of a method for generating a SQL command. According to an embodiment, the integration system comprises a SQL generator that transforms the configuration parameters defined by users, in the configuration interface, into SQL commands to retrieve data from EMR database. An SQL Executor (described below) is then responsible for executing the SQL commands.

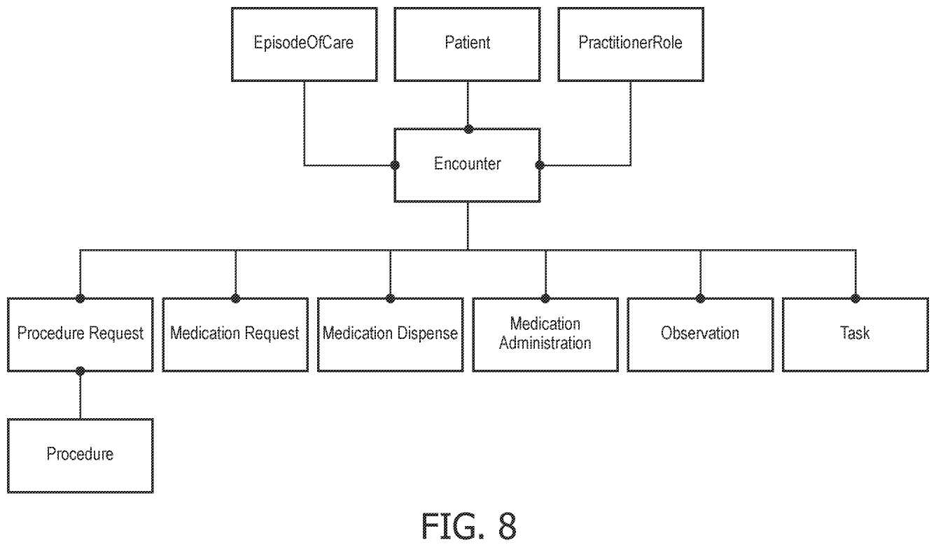

According to an embodiment, the first step of the method in FIG. 7 is to create a clause indicating which database columns should be retrieve. To do this, it uses the equivalence map information. According to an embodiment, the second step, which creates the FROM clause of the SQL command, is divided into two tasks. The first one includes the UNION clause, if the user had been selected more than one table for a given BPMN diagram element. The second includes the JOIN clause to link all tables involved in the SQL command. The logic of the join is defined according to the relationships established for FHIR resources. An example of relationships established for FHIR resources is shown in FIG. 8. According to an embodiment, the third step is to include the WHERE clause into SQL command, according to filters specified by user in the configuration interface.

According to an embodiment, the next two steps are to include the GROUP BY and ORDER BY clauses in the SQL command. The logic of including these clauses does not depend on user-defined parameters in the configuration interface, but according to the schema of the data, which is defined in the equivalence maps.

At step 112 of the method the integration system executes the generated SQL command to retrieve an electronic medical record from the EMR database. According to an embodiment, the integration system comprises a SQL executor responsible for retrieving EMR data by executing the SQL commands generated by the SQL generator. Unlike previous components, which may only be triggered at configuration time, the SQL executor can be running for all the time of the clinical pathways execution. According to an embodiment, the integration system comprises a synchronization engine that is responsible for orchestrating the process of recovery data from EMR and load them into CPMS database. Accordingly, the synchronization engine can trigger the SQL executor according to synchronization time defined by the user.

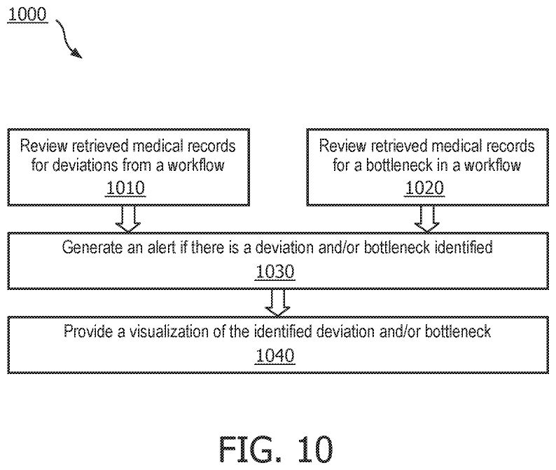

Referring to FIG. 10, in one embodiment, is a method 1100 for working with the results of at least a portion of the method described with regard to FIG. 1. For example, as described or otherwise envisioned herein, the integration system may analyze the retrieved data to look for deviations and/or bottlenecks, and may report that information to a user.

According to an embodiment, a mobile application system allows for tracking clinical pathways execution, presents the compliance status of each clinical pathway, and identifies process bottlenecks as soon as they occur. Thus, the system can provide an overview of the compliance status of all clinical pathways running in a hospital sector and allow the manager navigate to the patient level, can provide a mechanism able to identify deviations on the protocol and show these deviations in an adequate way to managers, and can provide a mechanism to identify or predict process bottlenecks and show it to a manager as soon as detected. Since it is a mobile application, a manager can track clinical pathways execution from anywhere.

At step 1010 of the method, according to an embodiment, the integration system analyzes, for at least one of the one or more of the plurality of patients, the retrieved electronic medical record for a deviation from a predefined clinical pathway workflow. The system may identify and/or report every deviation, or the system may be designed or programmed to identify and/or report only certain deviations, only deviations that meet certain predefined criteria, that are above a certain threshold, or otherwise satisfy a predefined parameter. Many ways of identifying deviations from a predefined clinical pathway workflow are possible.

At step 1020 of the method, according to an embodiment, the integration system analyzes, for at least one of the one or more of the plurality of patients, the retrieved electronic medical record for a bottleneck in a predefined clinical pathway workflow currently being executed for the at least one patient, or for multiple patients. The system may identify and/or report every bottleneck, or the system may be designed or programmed to identify and/or report only certain bottlenecks, only bottlenecks that meet certain predefined criteria, only bottlenecks that are above a certain threshold, or bottlenecks that otherwise satisfy a predefined parameter. Many ways of identifying bottlenecks from a predefined clinical pathway workflow are possible.

At step 1030 of the method, according to an embodiment, the integration system generates an alert if there is a deviation and/or bottleneck identified. The alert may simply be a trigger to proceed to step 1040 of the method. Optionally, the alert may be provided to a user via a user interface. Thus, the alert may be text, sound, haptic feedback, a visualization, text, email, or any other type of notification to a user. The alert may comprise information about the identified deviation and/or bottleneck, about the patient(s) involved, and any other information relevant to a user's analysis and/or understanding of the alert.

At step 1040 of the method, according to an embodiment, the integration system provides, via a user interface, a visualization of the identified deviation and/or bottleneck. The visualization can be any visualization that enables a user's analysis and/or understanding of the relevant information. Provided herein are several examples of visualizations of the identified deviation and/or bottleneck.

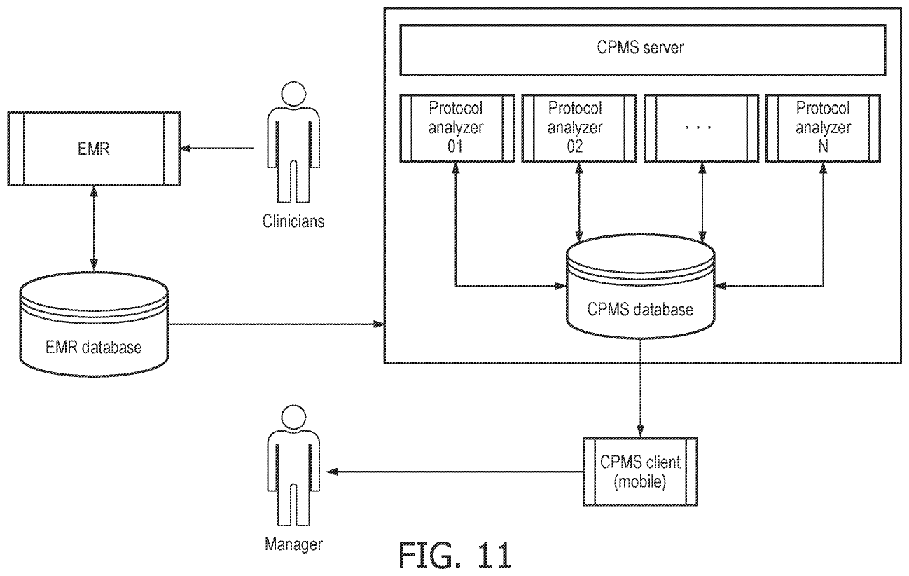

Referring to FIG. 11 is a schematic representation of a mobile application system for tracking clinical pathways execution. The mobile application system may be any of the systems described or otherwise envisioned herein. According to an embodiment, the system comprises an electronic medical record (EMR) database, a CPMS server, and a CPMS mobile client. The CPMS server implements a Protocol Analyzer application, which is an engine responsible for monitoring clinical pathways, by identifying deviations and bottlenecks. According to an embodiment, the Protocol Analyzer comprises four functional elements. The first one retrieves data from EMR, the second is responsible for identifying deviations, the third identifies process bottlenecks, and the fourth stores in the CPMS database the details regarding deviations and bottlenecks.

The CPMS Server can run one application instance for each institutional clinical protocol. Thus, if the hospital manages ten protocols, there can be ten instances running on the server. This strategy can be utilized because each protocol can have a different synchronization time. Some should be synchronized with EMR every minute, while others can be synchronized daily or even weekly. The mobile application responsible for presenting the deviations and bottlenecks information to department managers. This app shows the information produced by CPMS Server in a suitable format for help the fast decision making to solve or mitigate the problem.

According to an embodiment, the mobile application comprises four functional elements. The first one is responsible for synchronization with CPMS Server in order to retrieve information about deviations and bottlenecks. The other three components are responsible for presenting information to managers. The app can provide a consolidated view of all clinical pathways in execution and its conformance status, a view of the execution status of each patient included in the protocol, and/or a graphical view showing the bottlenecks of the process.

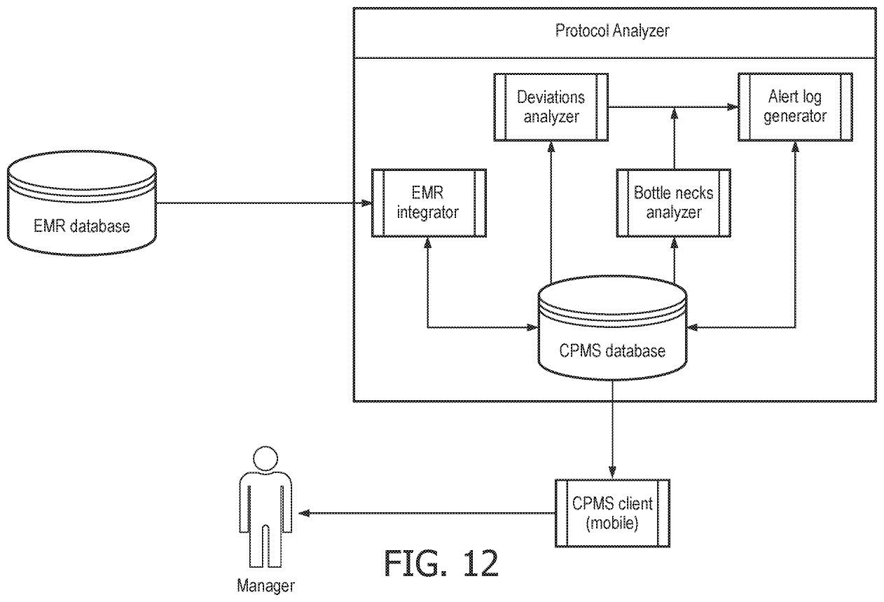

Referring to FIG. 12 is a schematic representation of a Protocol Analyzer to identify deviations and/or bottlenecks. A deviation may correspond to any nonconformity in the execution of the clinical pathway. The module can implement five different types of deviations, although other deviations can be identified: (1) tasks not executed (Missing Tasks Rules); (2) tasks executed out of order (Mandatory Order Rules); (3) tasks executed out of time (Target Time Rules); (4) tasks executed by an unauthorized role (Target Role Rules); and (5) excess or scarcity of resources (Target Quantity Rules). Each of these deviations types can be applied to each tasks of workflow. Thus, the user has the flexibility to define up to five or more compliance rules for each task.

The Protocol Analyzer can also identify bottlenecks in the process as well as potential bottlenecks, that is, those that have not yet occurred but are likely to occur. According to an embodiment, a bottleneck may be time and/or resources, among other possibilities. Time bottlenecks means that the execution time for a particular task, or the wait time between two tasks, is longer than usual. There are at least two possible ways to determine if the time is longer than normal: (a) if the execution time of the task is longer than the average, or (b) if it is greater than a predefined time for that task. The same logic is applicable to wait times.

Resource bottlenecks means the scarcity or lack of resources to perform a task, whether human or material resources such as medications, devices, and other material resources. The identification of resource bottlenecks is more complex than identifying time bottleneck because it requires a broader dataset, including a professional's timesheet, medication inventory, and/or other information. According to an embodiment, the system can identify resource bottlenecks such as a shortage of professionals to perform a task, a shortage or lack of medications, a shortage or lack of hospital supplies, a shortage or lack of beds, a shortage or lack of medical devices, and many other resource bottlenecks. A potential bottleneck can be identified from actual bottlenecks, once the occurrence of bottlenecks in a given task will affect all subsequent tasks.

According to an embodiment, the Protocol Analyzer comprises an alert log generator that stores information regarding deviations and bottlenecks identified by previous modules. The following data may be stored: type of occurrence (deviation or bottleneck); occurrence ID; description; date and time; task ID; resource ID (if applicable). These data can be stored into the CPMS database in a table (such as Alerts_Log), which can be used by the CPMS Client Application.

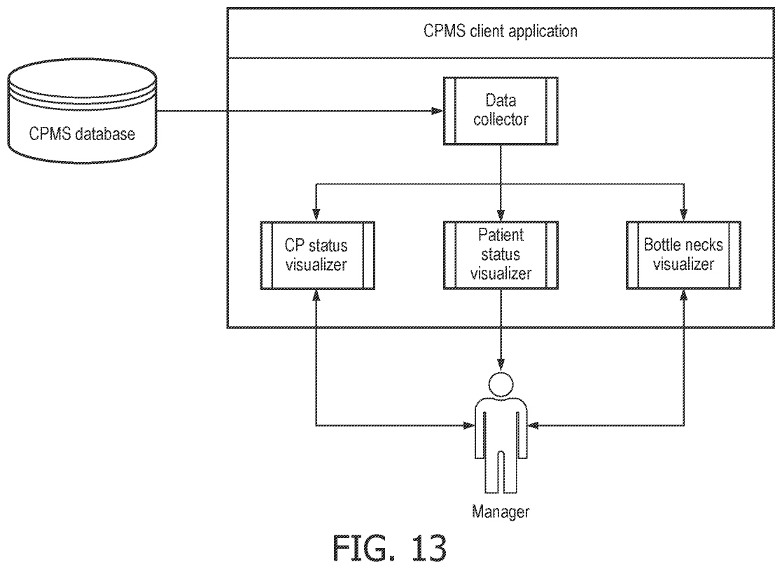

Referring to FIG. 13 is a schematic representation of a CPMS Client Application. According to an embodiment, the CPMS Client Application provides information to a user regarding an identified deviation and/or bottleneck. Therefore, the CPMS Client Application may comprise a CP status visualizer, patient status visualizer, and a bottleneck visualizer.

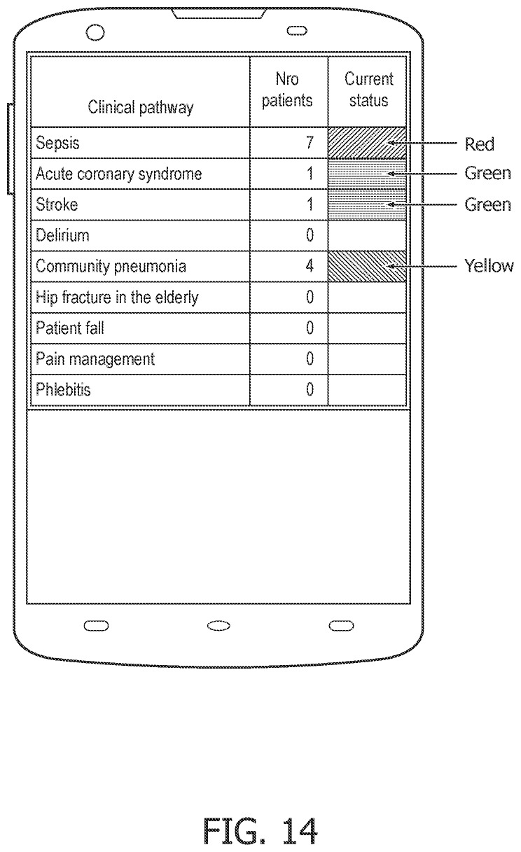

Referring to FIG. 14, in one embodiment, is a schematic representation of a CPMS Client Application visualization, which can be a mobile application main screen. It provides a consolidated view of all clinical pathways in execution and its compliance status. The CP Status Visualizer shown in FIG. 14 provides a simple view to the user, thereby allowing a fast identification of problems. The interface show the name of the protocol, the number of patients currently within the clinical pathway, and the execution overall status. This status is determined by colors, where green means that everything is in conformance, red indicates that some deviation or bottleneck has occurred in at least one case, and yellow indicates a potential deviation or bottleneck.

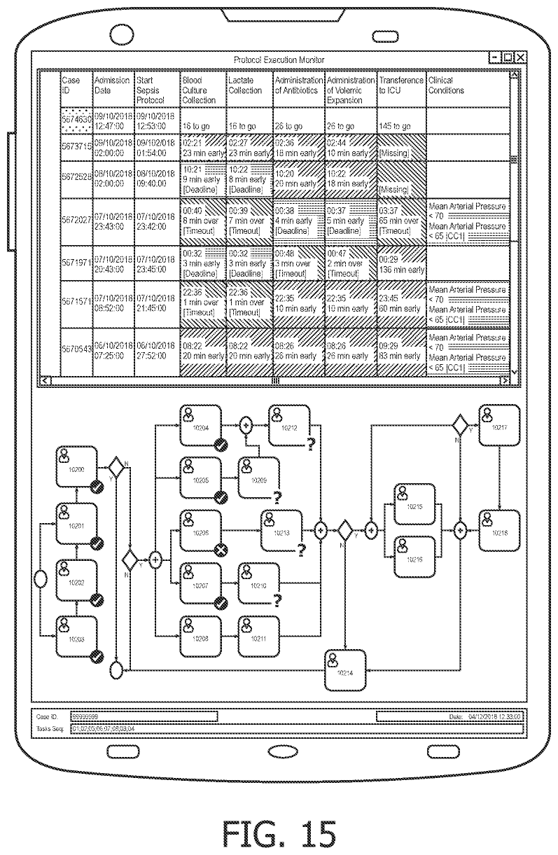

Referring to FIG. 15 is a schematic representation of a Patient Status Visualizer. According to an embodiment, the Patient Status Visualizer provides information to a user regarding a patient. Selection of a given protocol line on the main screen will trigger the list of patients contained in that protocol (FIG. 15, part A). Each column of the patient list corresponds to a task of clinical pathway. The compliance status of each task is presented using the same color system mentioned in the previous section. This view will allow the user to locate the patient and the exact task at which the deviation occurred. Selection of given patient in the list will display all details of the clinical pathway execution in a graphical format (FIG. 15, part B).

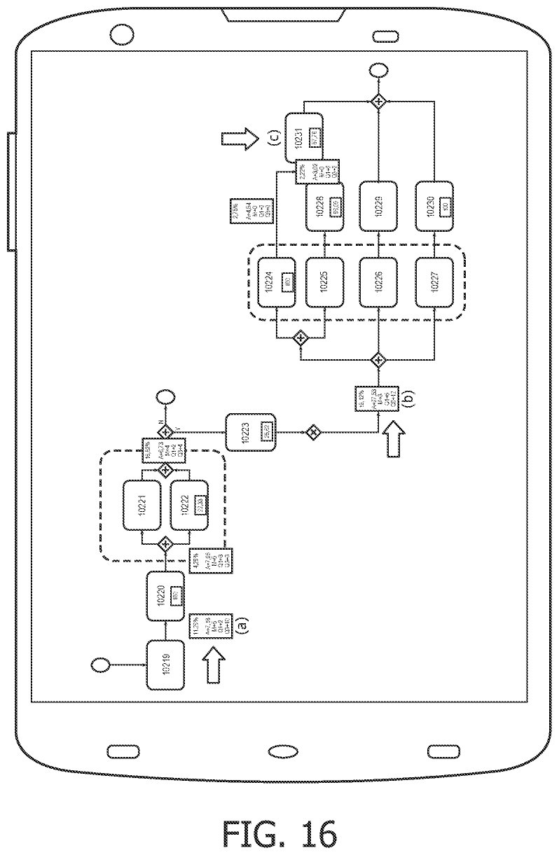

Referring to FIG. 16 is a schematic representation of a Bottlenecks Visualizer. According to an embodiment, the Bottlenecks Visualizer provides information to a user regarding one or more bottlenecks in a clinical pathway. The bottlenecks can be represented by boxes or other indication in a certain color, frame, or other visualization. In FIG. 16, three bottlenecks are indicated by the three large arrows.

According to an embodiment, the boxes located inside the task indicate a bottleneck in that task, while the boxes outside the tasks indicate a time bottleneck between two tasks. Considering FIG. 16, box (a) indicates a time bottleneck between the first and second tasks; and box (c) indicates a bottleneck (time or resource) in the last task. There can be text inside the box to provide information regarding the bottleneck, which can help the manager solve or mitigate the bottleneck.

Referring to FIG. 2 is a schematic representation of an integration system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, processor 220 may comprise, among other instructions or data, a CPMS database 262, equivalence map instructions 263, BPMN workflow instructions 264, configuration interface instructions 265, SQL generator instructions 266, SQL executor instructions 267, and/or alerting instructions 268.

According to an embodiment, CPMS database 262 comprises information about a current clinical pathway for each of the plurality of patients currently undergoing care. Alternatively, the CPMS database is hosted or stored remotely and is in communication the integration system and/or system 200.

According to an embodiment, equivalence map instructions 263 direct the system to generate an EMR equivalence map for the EMR database. According to an embodiment, the EMR equivalence map comprises a map of each of a plurality of objects in the EMR database to each of a plurality of elements in a standard data format. According to an embodiment, that standard data format can be fast healthcare interoperability resources (FHIR).

According to an embodiment, BPMN workflow instructions 264 direct the system to map each of one or more objects from the EMR equivalence map to each of one or more steps in one of a plurality of clinical pathway workflows. Therefore, according to an embodiment, the integration system comprises one or more digital workflows to which objects can be mapped. For example, the integration system may comprise a business process model and notation (BPMN) representation specifying one or more workflows or processes. The data transferred from the EMR to the CPMS can be related to the execution of tasks of a BPMN diagram. In this way, the integration system may allow the configuration of each element of the BPMN diagram.

According to an embodiment, configuration interface instructions 265 direct the system to provide a configuration interface that enables the user, without any programming effort, to configure the data integration between the ERM and the CPMS, thus integrating each clinical pathway BPMN element. According to an embodiment, the operation of the configuration interface consists of to select a BPMN element and to indicate which EMR database object should provide the data for that BPMN element. This operation is possible due to the equivalence map. Since the equivalence map defines all valid EMR tables and columns for a particular FHIR resource, the configuration interface may create a list with the database objects that the user can link to that BPMN element, according to an embodiment.

According to an embodiment, SQL generator instructions 266 direct the system to generate a SQL command. The SQL generator transforms the configuration parameters defined by users, in the configuration interface, into SQL commands to retrieve data from EMR database. According to an embodiment, SQL executor instructions 267 direct the system to execute the generated SQL command.

According to an embodiment, alerting instructions 268 direct the system to alert a user of the system to the identified one or more identified deviations and/or bottlenecks. According to an embodiment, the integration system 200 alerts the user via a user interface 240 of the system, and/or via other mechanisms. The system may alert the user via any mechanism for alert, including but not limited to a visual display, an audible notification, a text message, an email, a page, or any other method of notification. According to an embodiment, alerting instructions 268 direct the system to alert a user of the system to the identified one or more identified deviations and/or bottlenecks via the CPMS mobile client 280.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A computer-implemented method for integrating a clinical pathway management system with an electronic medical record database using an integration system, comprising:
providing a clinical pathways management system (CPMS) comprising a CPMS database, the CPMS database comprising information about a current clinical pathway for each of the plurality of patients currently undergoing care;
providing an electronic medical records (EMR) database comprising one or more medical records for each of a plurality of patients currently undergoing care in a clinical pathway;
generating in memory an EMR equivalence map for the EMR database, the EMR equivalence map comprising a map of each of a plurality of objects in the EMR database to each of a plurality of standard data format objects;
generating in memory a CPMS equivalence map for the CPMS database, the CPMS equivalence map comprising a map of each of a plurality of objects in the CPMS database to the plurality of standard data format objects;
mapping each of one or more objects from the EMR equivalence map to each of one or more steps in one of a plurality of clinical pathway workflows;
generating, using the map of each of one or more objects from the EMR equivalence map to each of one or more steps in one of a plurality of clinical pathway workflows, a SQL command related to an event in a clinical pathway workflow for one or more of the plurality of patients; and
executing the generated SQL command to retrieve an electronic medical record from the EMR database.

2. The method of claim 1, further comprising the steps of:
analyzing, for at least one of the one or more of the plurality of patients, the retrieved electronic medical record for a deviation from a predefined clinical pathway workflow;
generating an alert if there is a deviation identified; and
providing, via a user interface, a visualization of the identified deviation.

3. The method of claim 2, wherein the visualization of the identified deviation is provided via a mobile application.

4. The method of claim 3, wherein the identified deviation is provided via a mobile application utilizing a green, yellow, and red code.

5. The method of claim 1, further comprising the steps of:
analyzing, for at least one of the one or more of the plurality of patients, the retrieved electronic medical record for a bottleneck in a predefined clinical pathway workflow currently being executed for the at least one patient;
generating an alert if there is a bottleneck identified; and
providing, via a user interface, a visualization of the identified bottleneck.

6. The method of claim 5, wherein the visualization of the identified bottleneck is provided via a mobile application.

7. The method of claim 5, wherein the bottleneck is a time bottleneck and/or a resource bottleneck.

8. The method of claim 1, wherein the standard data format objects are fast healthcare interoperability resources (FHIR).

9. The method of claim 1, wherein the map of each of one or more objects from the EMR equivalence map to each of one or more steps in one of a plurality of clinical pathway workflows comprises Business Process Model and Notation (BPMN).

10. A system for integrating a clinical pathway management system with an electronic medical record database, comprising:
a clinical pathways management system (CPMS) comprising a CPMS database, the CPMS database comprising information about a current clinical pathway for each of the plurality of patients currently undergoing care;
an electronic medical records (EMR) database comprising one or more medical records for each of a plurality of patients currently undergoing care in a clinical pathway;
an EMR equivalence map for the EMR database, the EMR equivalence map comprising a map of each of a plurality of objects in the EMR database to each of a plurality of standard data format objects;
a CPMS equivalence map for the CPMS database, the CPMS equivalence map comprising a map of each of a plurality of objects in the CPMS database to the plurality of standard data format objects;
a processor configured to: (i) map each of one or more objects from the EMR equivalence map to each of one or more steps in one of a plurality of clinical pathway workflows; (ii) generate, using the map of each of one or more objects from the EMR equivalence map to each of one or more steps in one of a plurality of clinical pathway workflows, a SQL command related to an event in a clinical pathway workflow for one or more of the plurality of patients; and (iii) execute the generated SQL command to retrieve an electronic medical record from the EMR database.

11. The system of claim 10, wherein the processor is further configured to: analyze, for at least one of the one or more of the plurality of patients, the retrieved electronic medical record for a deviation from a predefined clinical pathway workflow; generate an alert if there is a deviation identified; and direct the system to provide, via a user interface, a visualization of the identified deviation.

12. The system of claim 11, wherein the system further comprises a mobile application with the user interface configured to provide the visualization of the identified deviation.

13. The system of claim 10, wherein the processor is further configured to: analyze, for at least one of the one or more of the plurality of patients, the retrieved electronic medical record for a bottleneck in a predefined clinical pathway workflow currently being executed for the at least one patient; generate an alert if there is a bottleneck identified; and direct the system to provide, via a user interface, a visualization of the identified bottleneck.

14. The system of claim 13, wherein the system further comprises a mobile application with the user interface configured to provide the visualization of the identified bottleneck.

15. The system of claim 10, wherein the standard data format objects are fast healthcare interoperability resources (FHIR).
